(19) United States
(12) Patent Application Publication
(10) Pub. No.: US 2010/0292771 A1
(43) Pub. Date: Nov. 18, 2010

(54) Endovascular stent graft system and guide system

(75) Inventor: Larry Paskar, Chesterfield, MO (US)

Correspondence Address: Gregory Upchurch, Suite 210, 1000 Des Peres Road, St. Louis, MO, 63131, US

(73) Assignee: SYNCARDIA SYSTEMS, INC, Tucson, AZ (US)

(21) Appl. No.: 12/454,420

(22) Filed: May 18, 2009

(51) Int. Cl. A61F 2/84

(52) U.S. Cl. 623/1.11

(57) ABSTRACT

Apparatus and method for inserting a stent graft limb branch into a stent graft body disposed in a living being, the stent graft body having a trunk and an opening for receiving said branch. A guide element is inserted through a vessel of the living being to the vicinity of the branch receiving opening of the stent graft body. The guide element is manipulated in three dimensions to form a shape corresponding to the orientation of the branch receiving opening with respect to the position of the vessel, so that the guide element shape is varied, in situ, to the required shape despite any abnormalities in the shape and orientation of the vessel. The guide element, after formation into the proper shape, is inserted into the branch receiving opening of the stent graft body. The stent graft limb branch is then fed over the guide element until the distal end of the limb branch is received in the branch receiving opening of the stent graft body. The guide element itself is also novel.

### CROSS REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

This invention pertains to medical devices and methods, and more particularly to such devices and methods relating to endovascular stent grafts.

2. Description of Related Art

An abdominal aortic aneurysm is a weakening, or balloon dilatation, of the major artery of the body, the aorta, often in the abdomen below the level of the renal arteries. This weakening of the wall of the aorta may be secondary to degenerative arteriosclerosis or predisposing genetic conditions, infection, inflammation or trauma. When the size of the aneurysm reaches a critical value of 5 cm in diameter, there is a real threat of aneurysm leak or rupture and a true medical emergency which can result in death due to hemorrhage.

Open abdominal surgical repair with placement or reinforcement of the dilated weakened segment of the aorta, using an artificial replacement graft segment, has long been practiced but is a difficult procedure for the patient with significant morbidity and mortality. In recent years, innovative minimally invasive surgical procedures have been developed. Rather than cutting through large areas of healthy tissue (such as making large incisions in the abdomen for gallbladder surgery for example), several small holes are made to allow passage of instruments through which the operative site can be viewed and the tissue, organ, etc. removed and/or treated. This spares the patient the major trauma of a large, slow healing open wound with attendant secondary complications related to slow recovery time, complications of longer surgery with its attendant risks. Recovery from laparoscopic cholecystectomy and other such minimally invasive procedures have proved revolutionary in medicine. Similar excellent results have recently been achieved using orifice surgery.

In keeping with the desire to develop a system for repairing an abdominal aortic aneurysm in a minimally invasive manner, stent grafts have been developed which can be delivered through a blood vessel to the aortic aneurysm, becoming a new conduit through which blood flows, so that the weakened aneurysmal segment is not exposed to high blood pressure and the attendant threat of aortic leak or rupture.

The stent graft can be delivered through the femoral artery on one side, and advanced to the weakened aneurysm, fixing the upper portion of the stent to the normal non-dilated infrarenal segment of the aorta, tracking as a new excluding artificial lumen segment within the weakened ballooned aneurysm, and continuing as branch components into the aortic bifurcation and normally branched native iliac arteries. The new co-axially placed artificial segment bridges the aorta from its normal infrarenal portion, mimicking the vascular anatomy branches into the left and right iliac arteries to isolate the weakened native aneurysmal portion from the threatening blood flow pressure by re-establishing normal flow through the channels of the artificial stent graft.

The prior art in the field provides relatively accurate self-delivery of the stent and creates strong, yet flexible stent grafts with good fixation and sealing proximally and distally of the stent graft. One step, which has not been optimized, is the method and means for easily passing a guidewire or such from the modular segment in the contralateral iliac artery into the short limb branch of the body of the stent graft, thereby allowing advancement of the modular segment tracking over various wires, or such, to ultimately telescope, seat and seal into the short limb, mimicking the native anatomy of the aorta and iliac bifurcation. An hour and-a-half procedure may become a three hour procedure while various shaped catheters, guidewires and guiding catheters are tried in an attempt to match the needed shape configuration and tip direction angulation and orientation with a complex anatomical pathway made more circuitous and serpiginous by inherent vascular dilatation and tortuosity of native vessels as well as the uncertainty of the anterior/posterior, medial/lateral relative positions of the short limb member position with respect to the modular short limb component.

What is needed and, heretofore, does not exist for this endograft stent procedure, is a medical tube that could be shaped and repeatedly reshaped, as necessary, at will, into a vast number of various curves with compound and/or complex distal end configurations which can be up-going, down-going out-of-plane as needed to allow custom formation of the appropriate distal end curve shape to match very complex anatomical and positional variations inherent in the patients undergoing these procedures.

It is known that many different shaped catheters have been developed and may be needed to get to a desired target area and remain in a stable, fixed position in the area to perform therapy on the desired precise site. What is needed is a system which allows the making of fine and coarse corrective adjustments to form the ideal contour shape and guarantee the precise tip position to match the very complex anatomy and variable orifice angle take-off positions allowing the tip to be navigated to the target, while at the same time, providing a contour shape abutting the opposing wall or acting as a stable platform in free space to allow precise passage of a payload, in this instance, a guidewire, bridging the modular segments which are to be telescoped, seated, sealed and joined together.

Currently, the native anatomy and resulting segment positions cannot be known or easily anticipated prior to the procedure to allow one to chose the appropriate medical tube shape, nor can the shape and precise position of the catheter tip be finely or coarsely adjusted during the procedure to accommodate for subtle variations which would allow for expeditious crossing of the modular segments. In fact the shape of the particular medical tube which is chosen at the beginning of the procedure could result in misalignment in all three dimensions.

What is needed is the appropriate tool for the appropriate job—converting a near-impossible procedure into a relatively easy procedure.

The ‘holy grail’ for precise navigation and negotiation of a medical tube through unknown, unpredictable, tortuous, serpiginous, body viscus, vessels, chambers, passages, spaces (potential and pathologic) is a guide system whose distal end could be configured and reconfigured ‘on the fly’ in real-time, in situ, in the body with imaging guided corrective feedback allowing coarse and fine readjustments in the shaped configurations and tip orientations with respect to its intended target as needed, to accommodate to the anatomical contour of the passage or space to be traversed. A system which would allow effective and repeated shaping and reshaping to form the precise shape necessary for the particular situation at hand would be extremely useful in this procedure.

Endovascular stent grafts for repair of more challenging thoracic aortic and suprarenal abdominal aortic aneurysms have been designed and developed. In addition to bypassing and internally reforming the weakened ballooned native channel and sealing it against systemic blood flow, modular components branching off of this stent must be provided to allow systemic flow through the artificial graft to supply major branch vessels which arise from the aorta to normally supply the brain and upper extremities as well as the thoracic and abdominal viscera which would otherwise be covered and, thereby, occluded by the walls of the artificial graft. This is not generally the case in the stent graft repair of the infrarenal abdominal aortic aneurysm where the bifurcation branches into the lower segments are provided for while other covered branch vessels serve vascular territories which have collateral and redundant natural branch blood supply through anastomoses with higher branch vessels.

In addition, the desired solution would allow such a minimally invasive procedure to be carried out to repair thoracic and suprarenal abdominal aortic aneurysms while maintaining communication with the central systemic blood flow of the aorta. (This could be accomplished by designing the body of the stent graft with branching modular components which could be delivered into, and sealed within, the native branch vessels—for example, the carotid and subclavian arteries.

Delivery of these branch elements could represent an equal or even more daunting challenge than delivery of the iliac stent module into the short limb of the infrarenal stent graft. Here the physician is faced with selectively catheterizing both the major stent branch vessel components and the major native branch vessel components, allowing passage of a guiding device to deliver the artificial component branches into the native branch vessels. This would require creative catheter shapes such as the up-going and down-going, as well as out-of-plane configurations developed to catheterize redundant or angled native brachial vessels in older individuals whose complex anatomical vascular take-off has developed as a result of aortic tortuosity and dilatation. The ability to mimic all of these shapes, as needed, in situ with one universal guide element would make stent grafting of major branch-bearing segments (thoracic and suprarenal abdominal) of the aorta much more efficient.

### BRIEF SUMMARY OF THE INVENTION

In a first aspect of the present invention, a method of inserting a stent graft limb branch into a stent graft body disposed in a living being (which stent graft body has a trunk and an opening for receiving the branch, includes the steps of inserting a guide element through a vessel of the living being to the vicinity of the branch receiving opening of the stent graft body, manipulating the guide element in three dimensions to form a shape corresponding to the orientation of the branch receiving opening with respect to the position of the vessel, whereby the guide element shape is varied, in situ, to the required shape despite any abnormalities in the shape and orientation of the vessel, inserting the guide element, after formation into the proper shape, into the branch receiving opening of the stent graft body; and feeding the stent graft limb branch over the guide element until the distal end of the limb branch is received in the branch receiving opening of the stent graft body.

In a second aspect of the present invention, an apparatus for inserting a stent graft limb branch into a stent graft body disposed in a living being includes a guide element sized to pass through a vessel of the living being to the vicinity of the branch receiving opening of the stent graft body, means for manipulating the guide element in three dimensions to form a shape corresponding to the orientation of the branch receiving opening with respect to the position of the vessel, whereby the guide element shape is varied, in situ, to the required shape despite any abnormalities in the shape and orientation of the vessel, The guide element, after formation into the proper shape, is of a size and shape to enter into the branch receiving opening of the stent graft body. Moreover, the said stent graft limb branch is movable over the guide element until the distal end of the limb branch is received in the branch receiving opening of the stent graft body.

In a third aspect of the present invention, a guide system for guiding medical tubes to sites in a living body, includes a guide element sized to pass through a vessel of the living being to the vicinity of the body at which a procedure is to be performed; and apparatus for manipulating the guide element in three dimensions to form a shape corresponding to the orientation of an operative area with respect to the position of the vessel, whereby the guide element shape is varied, in situ, to the required shape despite any abnormalities in the shape and orientation of the vessel.

### BRIEF DESCRIPTION OF THE DRAWINGS

FIGS. 1 and 1A are elevations showing the placement of a conventional stent graft.

FIGS. 2A and 2B illustrate some possible variation in the orientation of blood vessels in a living being.

FIGS. 3A-3C are plane slices also illustrating such variation.

FIG. 4 is a plane view illustrating misalignment in three dimensions of a vessel with the body of a stent graft.

FIGS. 5A and 5B are elevations showing up-going configurations of the guide element of the present invention.

FIGS. 6A and 6B are elevations showing down-going configurations of the guide element of the present invention.

FIGS. 7A-7C are elevations showing out-of-plane configurations of the guide element of the present invention.

Similar reference characters indicate similar parts throughout the various views of the drawings.

### DESCRIPTION OF THE PREFERRED EMBODIMENT(S)

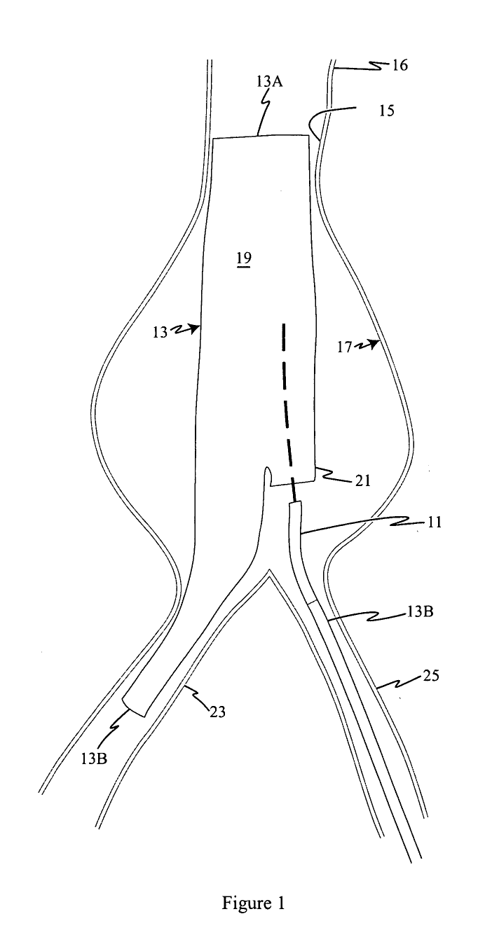

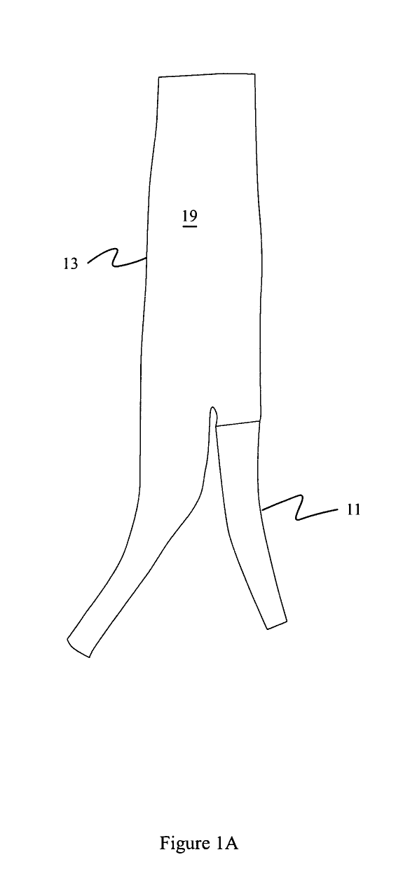

Turning now to the drawings, a method of the previous invention involves inserting a stent graft limb branch 11 into a stent graft body 13 disposed in a living being 15. In FIG. 1, the limb branch 11 is shown in a collapsed state, and the portion of the living body shown is the aorta 16 in the abdomen below the level of the renal arteries. The aneurysm in the aorta is labelled 17. The stent graft body 13 for ease of illustration is shown separated from the aorta walls, but it should be appreciated (and is known in the art) that the stent graft forms a tight seal in the conventional manner at both ends 13A and 13B. The stent graft body 13 has a trunk 19 and an opening 21 for receiving branch 11. For clarity (in FIG. 1A) limb branch 11 is shown in its expanded state in which it forms a unitary structure with the stent graft body 13.

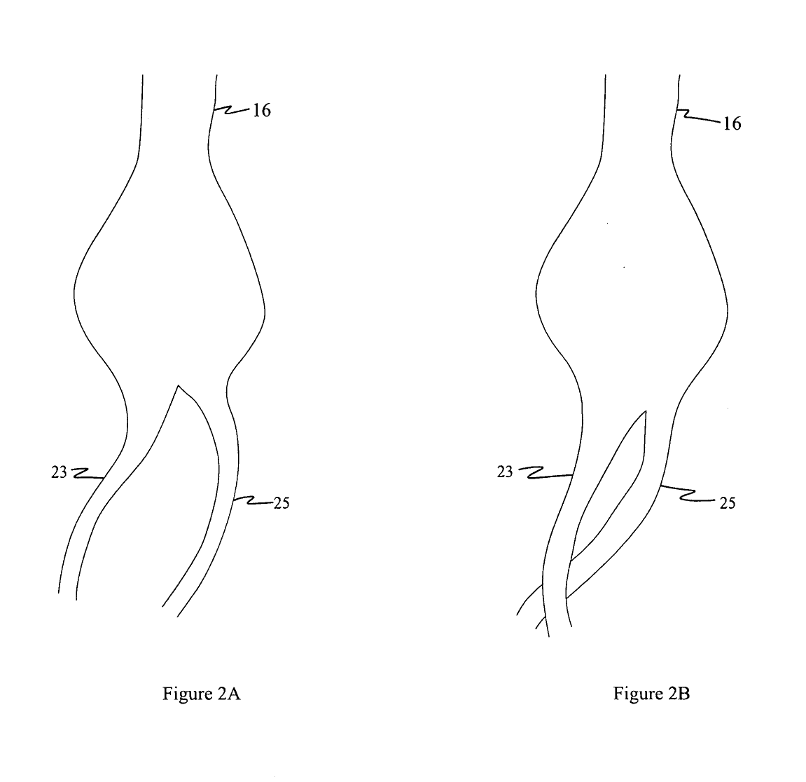

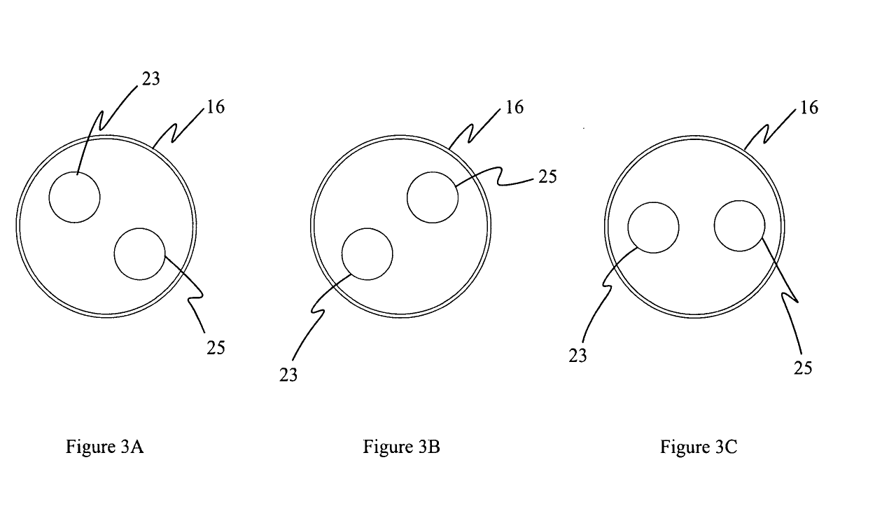

In FIG. 1 a fairly ideal shape of the aorta and iliac arteries configuration is illustrated. In actual living beings, the configuration is often far from ideal. For example, in FIGS. 2A and 2B, aorta 15 is illustrated with the left and right iliac arteries 23, 25 joining the aorta at various angles and with the iliac arteries themselves having various degrees of twists and turns. It should also be appreciated that the illustrations of FIGS. 1 and 2 are of necessity in two-dimensions, whereas the aorta and iliac arteries are three-dimensional structures with variation from ideal in all three dimensions. For example, in FIGS. 3A-3C a few of the vast numbers of possible configurations of the iliac arteries with respect to the aorta are illustrated at the point. As illustrated in FIGS. 2A and 2B, the relative positions of the iliac arteries change considerably as the aorta is approached. The result of all this variation is that the catheter (guide wire, tube, etc.) carrying the limb branch of the stent graft may exit the iliac artery 25 at any number of different angles in three dimensions. This makes accessing opening 21 extremely difficult.

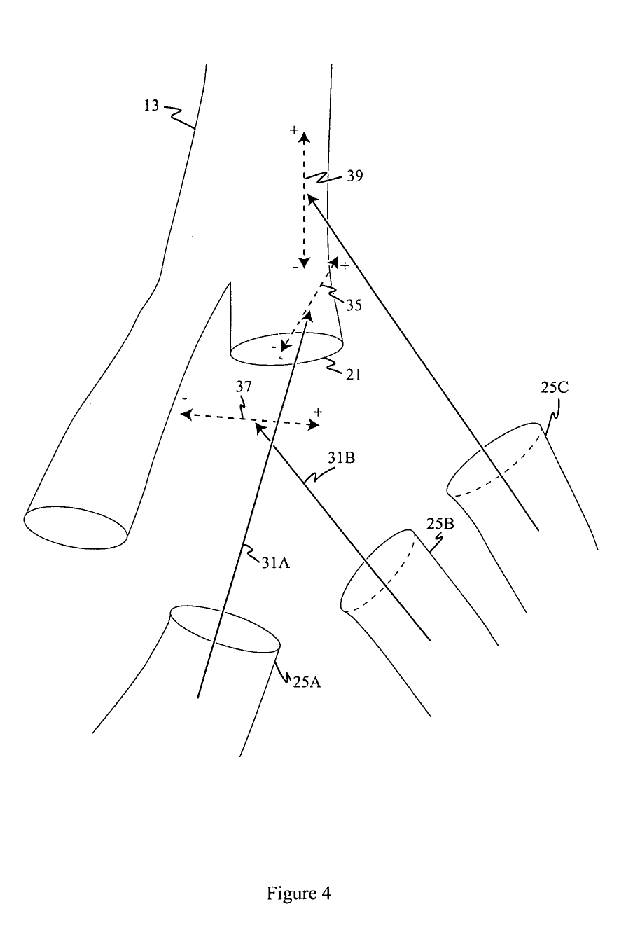

As shown in FIG. 4, the catheter 31 (shown in idealized form in FIG. 4) may miss opening 21 in any of three different ways (or any combination thereof) as a direct result of the relative position of the iliac artery 25 with respect to the placement of the stent graft body. In the situation indicated by the left-most catheter 31A emerging from iliac artery 25 (labeled 25A to distinguish it from two other possible positions 25B and 25C), the catheter 31A is disposed behind the body of stent graft 13. This represents an error in attempted placement in the direction into and out of the plane of the paper (indicated by the plus/minus arrow 35. Similarly, the middle catheter 31B emerging from the orientation illustrated by 25B, misses the opening 21 to the left. Variation in this plane is from side-to-side in the plane of the paper (plus/minus arrow 37). The rightmost illustration shows catheter 31B missing the opening in the vertical direction (plus/minus arrow 39). Of course, the actual misalignment in a given situation may be in all three dimensions, making insertion of the limb branch an extremely trying and time-consuming effort.

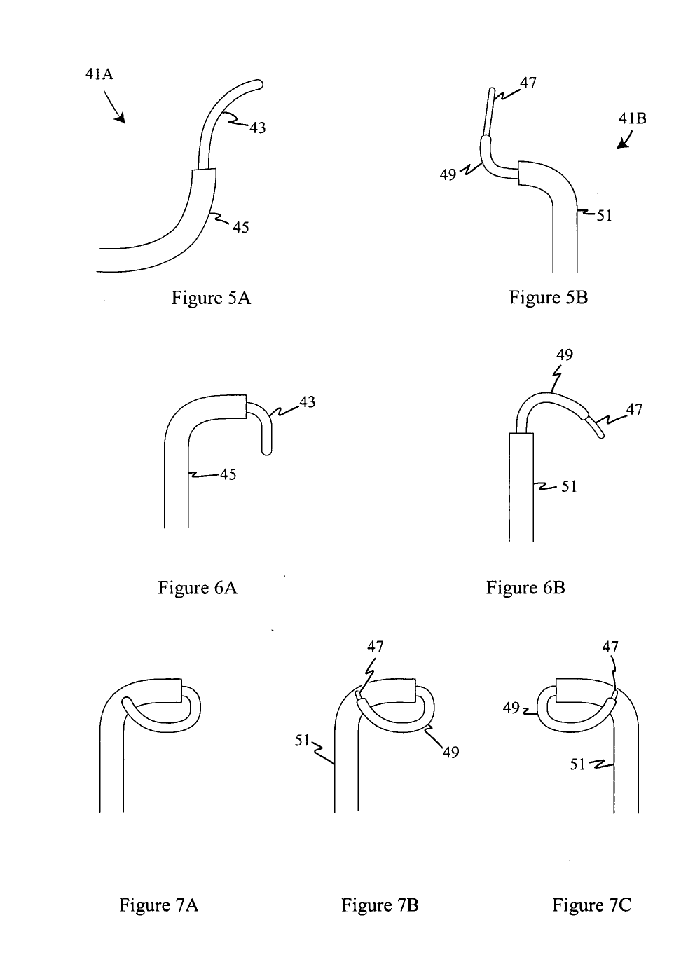

The present invention solves this problem by inserting a guide element (such as a guide wire or a guide catheter through a vessel of the living being to the vicinity of the branch receiving opening of the stent graft body. As illustrated in FIGS. 5A and 5B, the guide element may be two- or three-part. In FIG. 5A, the two-part guide element 41A is shown to have an inner curved or curvable element 43 (such as a conventional guide wire) which interacts with an outer curved or curvable medical tube 45. The inner element and outer tube are movable rotationally and translationally with respect to each other so that the curved or curvable distal end portions thereof may interact to form a vast multitude of possible shapes, including the exact shape needed to access opening 21 in the stent graft body 13. In FIG. 5B, the three-part guide element 41B is shown to have a straight inner element 47 (preferably a conventional guide wire) which is basically pointed in the correct direction by the interaction of a curved or curvable inner medical tube 49 and a curved or curvable outer medical tube 51. Inner element 47 is movable translationally with respect to the inner medical tube 49, and the inner and outer medical tubes 49 and 51 are movable both rotationally and translationally with respect to each other to form the vast variety of shapes described above. Once the necessary shape is formed, the inner element 47 is moved translationally with respect to the inner medical tube 49 to access the opening 21 in the stent graft body 13.

In either case, the guide element 41 is thus manipulated in three dimensions to form a shape corresponding to the orientation of the branch receiving opening 21 with respect to the position of the vessel 25, so that the guide element shape is varied, in situ, to the required shape despite any abnormalities in the shape and orientation of the vessel. Thereupon the guide element 41, after formation into the proper shape, is inserted into the branch receiving opening 21 of the stent graft body 13. Stent graft limb branch 11 is then fed over the guide element until the distal end of the limb branch is received in the branch receiving opening of the stent graft body.

Among the various shapes that can be achieved by the present method are the up-going shapes shown in FIGS. 5A and 5B, as well as down-going shapes (see FIG. 6) and out-of-plane shapes (see FIG. 7). An up-going shape is one in which the extreme distal end of the guide element points in the direction which corresponds to the direction of advancement of the catheter into the body. Similarly, a down-going shape is one in which the extreme distal end of the guide element points in a direction opposite to the direction of advancement of the catheter into the body. An out-of-plane shape is one which is out of the plane defined by the curved distal end portion of the outer medical tube. It should be understood that the particular up-going, down-going, and out-of-plane shapes are illustrative only, and that a vast number of such shapes may be generated as needed by the present invention. Of particular interest is the fact that the out-of-plane shapes (FIG. 7) may be either clockwise out-of-plane shapes (e.g., FIG. 7B) or counter-clockwise out-of-plane shapes (FIG. 7C) as the circumstances require. All these shapes are achieved by simply moving the curved/curvable elements translationally and rotationally with respect to each other. It should also be noted that these shapes are three-dimensional, so that (for example) up-going, out-of-plane or down-going, out-of-plane shapes may be made as well.

The guide element with its outer tube 45 or 51 also provides a stable platform for the feeding of the stent graft limb branch 11. This can be done under either fluoroscopic or optical imaging. Although a particular stent graft and artery have been illustrated, the present invention is not limited in that way. For example, the stent graft body may be disposed in an abdominal artery such as a suprarenal abdominal artery or in a thoracic artery, or in any other artery in which stent grafts are placed.

Nor is the invention limited to the placement of stent grafts and stent graft limbs. The guide element disclosed herein is of general usefulness and may be used in those cases in which a conventional guide wire is typically used.

What is claimed is:

1. A method of inserting a stent graft limb branch into a stent graft body disposed in a living being, said stent graft body having a trunk and an opening for receiving said branch, comprising:
inserting a guide element through a vessel of the living being to the vicinity of the branch receiving opening of the stent graft body;
manipulating the guide element in three dimensions to form a shape corresponding to the orientation of the branch receiving opening with respect to the position of the vessel, whereby the guide element shape is varied, in situ, to the required shape despite any abnormalities in the shape and orientation of the vessel;
inserting the guide element, after formation into the proper shape, into the branch receiving opening of the stent graft body;
feeding the stent graft limb branch over the guide element until the distal end of the limb branch is received in the branch receiving opening of the stent graft body.

2. The method as set forth in claim 1 wherein the manipulating step includes forming the guide element into an up-going shape.

3. The method as set forth in claim 1 wherein the manipulating step includes forming the guide element into a down-going shape.

4. The method as set forth in claim 1 wherein the manipulating step includes forming the guide element into an out-of-plane shape.

5. The method as set forth in claim 4 wherein the manipulating step includes forming the guide element into a counter-clockwise out-of-plane shape.

6. The method as set forth in claim 4 wherein the manipulating step includes forming the guide element into a clockwise out-of-plane shape.

7. The method as set forth in claim 1 wherein the manipulating step includes forming the guide element into an up-going, out-of-plane shape.

8. The method as set forth in claim 1 wherein the manipulating step includes forming the guide element into a down-going, out-of-plane shape.

9. The method as set forth in claim 1 wherein the guide element includes first and second elements with distal end portions capable of assuming curves, said first and second elements being coaxially disposed and longitudinally movable with respect to each other.

10. The method as set forth in claim 1 wherein the guide element provides a stable platform for the feeding of the stent graft limb branch.

11. The method as set forth in claim 1 wherein the manipulating of the guide element occurs under fluoroscopic imaging.

12. The method as set forth in claim 1 wherein the manipulating for the guide element occurs under optical imaging.

13. The method as set forth in claim 1 wherein the stent graft body is disposed in an abdominal artery.

14. The method as set forth in claim 1 wherein the stent graft body is disposed in a suprarenal abdominal artery.

15. The method as set forth in claim 1 wherein the stent graft body is disposed in a thoracic artery.

16. Apparatus for inserting a stent graft limb branch into a stent graft body disposed in a living being, said stent graft body having a trunk and an opening for receiving said branch, comprising:
a guide element sized to pass through a vessel of the living being to the vicinity of the branch receiving opening of the stent graft body;
means for manipulating the guide element in three dimensions to form a shape corresponding to the orientation of the branch receiving opening with respect to the position of the vessel, whereby the guide element shape is varied, in situ, to the required shape despite any abnormalities in the shape and orientation of the vessel;
said the guide element, after formation into the proper shape, being of a size and shape to enter into the branch receiving opening of the stent graft body;
said stent graft limb branch being movable over the guide element until the distal end of the limb branch is received in the branch receiving opening of the stent graft body.

17. The apparatus as set forth in claim 16 wherein the guide element is a guide wire with a curved distal end portion and the means for manipulating includes a catheter with a distal end portion capable of assuming a curve such that the guide wire curved distal end portion and the catheter distal end portion interact to form the required three-dimensional shape.

18. The apparatus as set forth in claim 16 wherein the guide element is a straight guide wire and the means for manipulating includes a first catheter with a distal end portion capable of assuming a curve and a second catheter with a distal end portion capable of assuming a curve such that the distal end portions of the first and second catheters interact to form the required three-dimensional shape, the guide wire being extendible from the first and second catheters.

19. A guide system for guiding medical tubes to sites in a living body, comprising:
a guide element sized to pass through a vessel of the living being to the vicinity of the body at which a procedure is to be performed; and
means for manipulating the guide element in three dimensions to form a shape corresponding to the orientation of an operative area with respect to the position of the vessel, whereby the guide element shape is varied, in situ, to the required shape despite any abnormalities in the shape and orientation of the vessel.

20. The apparatus as set forth in claim 19 wherein the guide element is a guide wire with a curved distal end portion and the means for manipulating includes a catheter with a distal end portion capable of assuming a curve such that the guide wire curved distal end portion and the catheter distal end portion interact to form the required three-dimensional shape.

21. The apparatus as set forth in claim 19 wherein the guide element is a straight guide wire and the means for manipulating includes a first catheter with a distal end portion capable of assuming a curve and a second catheter with a distal end portion capable of assuming a curve such that the distal end portions of the first and second catheters interact to form the required three-dimensional shape, the guide wire being extendible from the first and second catheters.
